(19) United States
(12) Patent Application Publication
(10) Pub. No.: US 2018/0264167 A1
(43) Pub. Date: Sep. 20, 2018

(54) CEMENT-FORMING COMPOSITIONS, APATITE CEMENTS, IMPLANTS AND METHODS FOR CORRECTING BONE DEFECTS

(71) Applicant: OssDsign AB, Uppsala (SE)

(72) Inventor: Håkan ENGQVIST, Uppsala (SE)

(73) Assignee: OssDsign AB, Uppsala (SE)

(21) Appl. No.: 15/761,979

(22) PCT Filed: Sep. 22, 2016
(86) PCT No.: PCT/IB2016/055677

§ 371 Date: Mar. 21, 2018

Related U.S. Application Data
(60) Provisional application No. 62/222,252, filed on Sep. 23, 2015.

(51) Int. Cl. A61L 27/12; C04B 28/34; C04B 14/48; A61L 27/06; A61L 27/56; A61L 27/50

(52) U.S. Cl. CPC A61L 27/12; C04B 28/344; C04B 14/48; C04B 2111/00836; A61L 27/56; A61L 27/50; A61L 27/06

(57) ABSTRACT

A calcium phosphate apatite cement-forming composition comprises an apatite-forming calcium-based precursor powder and from 1 to 30 wt %, based on the weight of the precursor powder, of dicalcium pyrophosphate powder or or sodium pyrophosphate powder. Apatite cements formed form such compositions may be used in implants for correcting bone defects. Methods for bone defect repair employ implants formed from such apatite cements and slow implant resorption and/or improve in vivo bone induction in a patient.

### FIELD OF THE INVENTION

The invention relates to cement-forming compositions, apatite cements, implants, and methods for correcting bone defects.

### BACKGROUND OF THE INVENTION

Bone tissue defects that cannot heal via tissue regeneration can be filled using autograph, allograph or synthetic scaffold materials. For large defects, e.g. defects in the cranium or in long bones, healing of bone defects can be especially difficult. A wealth of bioceramic formulations and delivery forms have been suggested for use as bone void filler materials. Examples of bone void fillers include calcium phosphate cements, e.g. apatite and brushite based cements, powders and granules, of, e.g., tricalcium phosphates, such as β-TCP and α-TCP, and tetracalcium phosphate. Delivery forms include injectable forms and granules packed directly into an open bone defect. Injectable cements have been proposed both as premixed versions and as formulations to be mixed in the operating room. One major drawback with the current suggested material formulations is their relatively low bone induction capability. This is especially important in repair of large and complex bone defects, as in the cranium. Some bioceramic formulations which have been reported as having an ability to induce bone formation include hydroxyapatite (porous), biphasic calcium phosphate ceramics, tricalcium phosphate ceramic, calcium pyrophosphate and apatite cement formulations. However, conventional clinically used materials are typically either too chemically stable, i.e., they do not exhibit any or too little resorption, or they resorb too fast, which can result in an open bone defect in vivo. Tailoring of the resorption rate, i.e., release of ions, to match the formation of new bone and release of ions that stimulate bone formation would be a fruitful development. Bone induction properties would allow the synthetic material to compete with autologous bone to a greater extent. However, bone induction capability of calcium phosphate formulations has been very difficult to combine with a tailored resorption rate and a material handling technique that facilitates industrial use of the materials, e.g. in the operating room and/or for moulding of complex shapes.

Accordingly, there is an unmet need for a material that has a slow and optimal resorption rate in vivo and/or induces bone formation, and is easily handled in the operating room and/or when moulding complex shaped implants.

### SUMMARY OF THE INVENTION

This invention is directed to compositions and methods that fulfil one or more of these unmet needs.

In one embodiment, the invention is directed to calcium phosphate cement-forming compositions which comprise an apatite-forming calcium-based precursor powder and, optionally, a non-aqueous water-miscible liquid.

In one specific embodiment, the apatite-forming calcium-based precursor powder comprises α-tricalcium phosphate (α-Ca₃(PO₄)₂), and from 1 to 30 wt %, based on the weight of the precursor powder, of dicalcium pyrophosphate (Ca₂P₂O₇, also referred to herein as calcium pyrophosphate) powder or sodium pyrophosphate (Na₄O₂P₇ or Na₄P₇O₂, also known as tetrasodium pyrophosphate) powder.

In a second specific embodiment, the apatite-forming calcium-based precursor powder comprises tetracalcium phosphate (Ca₄(PO₄)₂O), and from 1 to 30 wt %, based on the weight of the precursor powder, of dicalcium pyrophosphate or sodium pyrophosphate and is adapted to be mixed with an aqueous liquid or exposed to an aqueous liquid to achieve hardening.

This invention is also directed to apatite cements formed form such calcium phosphate cement-forming compositions and to apatite cements comprising from 1 to 30 wt % of dicalcium pyrophosphate or sodium pyrophosphate.

This invention is also directed to implants comprising an apatite cement, wherein the apatite cement comprises from 1 to 30 wt % of dicalcium pyrophosphate or sodium pyrophosphate. In a more specific embodiment, the implants comprise a wire or mesh and one or a plurality of ceramic tiles moulded on the wire or mesh, wherein the ceramic tiles are formed of an apatite cement comprising from 1 to 30 wt % of β-dicalcium pyrophosphate or sodium pyrophosphate. In a specific embodiment, the wire or mesh is formed of titanium. In another embodiment, the implant is provided in the form of hardened granules which may be placed in a patient's body.

This invention is also directed to methods of correcting bone defects. In one embodiment, such methods comprise slowing implant resorption in a bone defect repair in a patient by providing the patient with an implant formed of an apatite cement comprising from 1 to 30 wt % of dicalcium pyrophosphate or sodium pyrophosphate. In another embodiment, such methods comprise providing improved bone induction in a bone defect repair in a patient by providing the patient with an implant formed of an apatite cement comprising from 1 to 30 wt % of dicalcium pyrophosphate or sodium pyrophosphate. In another specific embodiment, these methods employ β-dicalcium pyrophosphate.

This invention is also directed to implants which slow bone resorption and/or improve bone induction in a bone defect repair in a patient, wherein the implant is formed of an apatite composition comprising from 1 to 30 wt % of dicalcium pyrophosphate or sodium pyrophosphate. In another embodiment, these implants employ β-dicalcium pyrophosphate.

The cement-forming compositions, cements, implants and methods of the invention are advantageous in that they provide implants which have optimal resorption rates in vivo and/or induce bone formation, and are easily handled in the operating room or when moulding complex shaped implants. These and additional embodiments and advantages of the invention will be more apparent in view of the Detailed Description.

### BRIEF DESCRIPTION OF THE DRAWINGS

Various embodiments of the invention will be more fully understood in view of the drawing in which:

FIG. 1 shows one embodiment of an implant structure according to the present invention.

### DETAILED DESCRIPTION

The present invention is directed to calcium phosphate apatite cement-forming compositions, apatite-forming calcium phosphate-based precursor powders for forming apatite cements, and apatite cements. The invention is also directed to implants formed of apatite cements and methods for correcting bone defects with apatite cement implants.

The calcium phosphate apatite cement-forming compositions comprise a apatite-forming calcium-based precursor powder. In one specific embodiment, the apatite-forming calcium-based precursor powder comprises α-tricalcium phosphate and/or tetracalcium phosphate, and from 1 to 30 wt %, based on the weight of the precursor powder, of dicalcium pyrophosphate (also referred to herein as calcium pyrophosphate) powder or sodium pyrophosphate powder. In respective specific embodiments, the calcium-based precursor powder comprises α-tricalcium phosphate, tetracalcium phosphate, and a mixture of α-tricalcium phosphate and tetracalcium phosphate.

In one specific embodiment, the apatite-forming calcium-based precursor powder comprises α-tricalcium phosphate and/or tetracalcium phosphate, and calcium pyrophosphate. In one embodiment, the precursor powder comprises from about 70 to 99 wt % of α-tricalcium phosphate and/or tetracalcium phosphate and from 1 to 30 wt % of dicalcium pyrophosphate powder, based on the weight of the precursor powder.

In another specific embodiment, the apatite-forming calcium-based precursor powder comprises α-tricalcium phosphate and/or tetracalcium phosphate, and sodium pyrophosphate. In one embodiment, the precursor powder comprises from about 70 to 99 wt % of α-tricalcium phosphate and/or tetracalcium phosphate and from 1 to 30 wt % of sodium pyrophosphate powder, based on the weight of the precursor powder.

In one embodiment, the apatite cement-forming compositions comprise the precursor powder as described and a non-aqueous water-miscible liquid. The precursor powder to liquid (wt/vol, i.e., g/ml) ratio may be from about 1 to 7, or more specifically, from about 2 to 6 in the cement compositions, or from about 2.5 to about 5, or from about 3 to about 4.5, for better handling and mechanical strength. The nonaqueous liquid facilitates handling and use, without premature hardening of the cement-forming compositions. Examples of the non-aqueous water-miscible liquid employed in embodiments according to the invention include, but are not limited to, glycerol and related liquids, compounds and derivates (substances derived from non-aqueous water-miscible substances), substitutes (substances where part of the chemical structure has been substituted with another chemical structure) and the like. The purpose of the non-aqueous water-miscible liquid is to give a longer working time during the moulding of the implant or during injection in the operating room (if used as an injectable cement). Certain alcohols may also be suitable for use as such a liquid. In specific embodiments, the liquid is selected from glycerol, propylene glycol, poly(propylene glycol), poly(ethylene glycol) and combinations thereof. In specific embodiments containing the non-aqueous liquid, the composition liquid may be entirely non-aqueous or may be partly aqueous, i.e., containing <20 vol % water, or less than 10 vol % water, in the mixing liquid.

In another embodiment, the calcium phosphate cement-forming compositions comprise an apatite-forming calcium-based precursor powder as described above and may be mixed with an aqueous liquid or exposed to an aqueous liquid to achieve hardening. The liquid can be water or a water-based mixture. In one embodiment, the precursor powder composition is chosen to obtain a setting time above about 30 minutes. The cement-forming precursor powder is mixed with and/or exposed to water to achieve setting of the cement. This can be conducted for producing pre-formed implants or at the time of surgery for in vivo setting of the cement.

In certain embodiments employing sodium pyrophosphate, especially higher amounts of sodium pyrophosphate, the setting time to achieve a hardened cement may be increased to several hours. In the event that a shorter setting time is desired, heat can be applied to the composition to obtain a faster hardening time.

In specific embodiments, the precursor powder compositions and/or the apatite cement compositions according to the invention comprise from 1 to 30 wt % of dicalcium pyrophosphate or sodium pyrophosphate. In further embodiments, the dicalcium pyrophosphate or sodium pyrophosphate comprises from 1 to 10 wt %, from 2 to 10 wt %, from 3 to 10 wt %, from 4 to 10 wt %, from 5 to 10 wt %, from 6 to 10 wt %, from 7 to 10 wt %, or from 8 to 10 wt %, of the precursor powder and/or the apatite cement composition. In further embodiments, the dicalcium pyrophosphate or sodium pyrophosphate comprises from 1 to 5 wt %, from 2 to 5 wt %, from 3 to 5 wt %, or from 4 to 5 wt % of the precursor powder and/or the apatite cement composition.

In further embodiments, the dicalcium pyrophosphate or sodium pyrophosphate comprises from 1 to 15 wt %, from 2 to 15 wt %, from 3 to 15 wt %, from 4 to 15 wt %, from 5 to 15 wt %, from 6 to 15 wt %, from 7 to 15 wt %, from 8 to 15 wt %, from 9 to 15 wt %, from 10 to 15 wt %, from 11 to 15 wt %, or from 12 to 15 wt %, of the precursor powder and/or the apatite cement composition. In further embodiments, the dicalcium pyrophosphate or sodium pyrophosphate comprises from 1 to 20 wt %, from 2 to 20 wt %, from 3 to 20 wt %, from 4 to 20 wt %, from 5 to 20 wt %, from 6 to 20 wt %, from 7 to 20 wt %, from 8 to 20 wt %, from 9 to 20 wt %, from 10 to 20 wt %, from 11 to 20 wt %, from 12 to 20 wt %, or from 15 to 20 wt %, of the precursor powder and/or the apatite cement composition. In further embodiments, the dicalcium pyrophosphate or sodium pyrophosphate comprises from 1 to 25 wt %, from 2 to 25 wt %, from 3 to 25 wt %, from 4 to 25 wt %, from 5 to 25 wt %, from 6 to 25 wt %, from 7 to 25 wt %, from 8 to 25 wt %, from 9 to 25 wt %, from 10 to 25 wt %, from 11 to 25 wt %, from 12 to 25 wt %, from 13 to 25 wt %, from 14 to 25 wt %, from 15 to 25 wt %, or from 20 to 25 wt %, of the precursor powder and/or the apatite cement composition. In further embodiments, the dicalcium pyrophosphate or sodium pyrophosphate comprises from 2 to 30 wt %, from 3 to 30 wt %, from 4 to 30 wt %, from 5 to 30 wt %, from 6 to 30 wt %, from 7 to 30 wt %, from 8 to 30 wt %, from 9 to 30 wt %, from 10 to 30 wt %, from 11 to 30 wt %, from 12 to 30 wt %, from 13 to 30 wt %, from 14 to 30 wt %, from 15 to 30 wt %, from 16 to 30 wt %, from 17 to 30 wt %, from 18 to 30 wt %, from 19 to 30 wt %, from 20 to 30 wt %, from 21 to 30 wt %, from 22 to 30 wt %, from 23 to 30 wt %, from 24 to 30 wt %, or from 25 to 30 wt %, of the precursor powder and/or the apatite cement composition.

In any of these described embodiments, reference to calcium dipyrophosphate or sodium pyrophosphate includes mixtures of calcium dipyrophosphate and sodium pyrophosphate, in any proportion of components. For example, such mixtures may comprise from 1:99 to 99:1 weight ratio of calcium dipyrophosphate to sodium pyrophosphate, from 10:90 to 90:10 weight ratio of calcium dipyrophosphate to sodium pyrophosphate, or from 25:75 to 75:25 weight ratio of calcium dipyrophosphate to sodium pyrophosphate.

In any of the embodiments disclosed herein, the dicalcium pyrophosphate may comprise alpha-dicalcium pyrophosphate, beta-dicalcium pyrophosphate and/or gamma-calcium pyrophosphate. In specific embodiments, the dicalcium pyrophosphate comprises beta-dicalcium pyrophosphate. In other specific embodiments, the dicalcium pyrophosphate comprises alpha-dicalcium pyrophosphate. In other specific embodiments, the dicalcium pyrophosphate comprises gamma-dicalcium pyrophosphate.

The dicalcium pyrophosphate may be added to the calcium phosphate precursor powder or, alternatively, the dicalcium pyrophosphate may be formed during the formation of the precursor powder, for example, by addition of CaCO₃ in the formation of α-tricalcium phosphate or tetracalcium phosphate. For example, a solid-state diffusion controlled synthesis may be employed wherein pyrophosphate is formed simultaneously with α-TCP, β-TCP and/or TTCP. For TCP formation, the reaction proceeds as: CaCO₃+Ca₂P₂O₇→Ca₃(PO₄)₂+CO₂ and for TTCP formation, the reaction proceeds as: 2CaHPO₄+2CaCO₃→Ca₄(PO₄)20 +CO₂+H₂O. For the formation of TTCP and α-TCP, rapid cooling from high temperatures are needed, as the phases are not stable at low temperatures below about 1000° C. The pyrophosphate content will be controlled via adding CaCO₃ in varying amounts to the starting powder. Formation of the pyrophosphate during the formation of the precursor powder results in co-nucleation of grains, which results in a stronger, hardened cement and enables a slow release as compared with addition of pyrophosphate as a separate powder to a precursor powder mix. Pyrophosphate can nucleate during the reaction based upon the amount of calcium which is available, i.e., based on addition of a non-stoichiometric amount of calcium to the raw material composition.

The apatite cements contain a majority, i.e., greater than 50 wt %, of apatite cement. In specific embodiments, the apatite cements contain at least 55 wt %, at least 60 wt %, at least 65 wt %, at least 70 wt %, at least 75 wt %, at least 80 wt %, at least 85 wt %, or at least 90 wt %, apatite. In additional embodiments, the apatite cements contain a minor amount of β-tricalcium phosphate. In more specific embodiments, the apatite cements contain from about 1 to 15 wt %, 1 to 10 wt %, or 2 to 20 wt %, of β-tricalcium phosphate.

Thus, specific embodiments of the apatite cements described herein comprise greater than 70 wt % or greater than 80 wt % apatite, 1 to 15 wt % or 1 to 10 wt % β-tricalcium phosphate, and less than 30 wt %, 1 to 20 wt % or 1 to 15 wt % dicalcium pyrophosphate or sodium pyrophosphate, or, more specifically, β-dicalcium pyrophosphate.

In certain alternate embodiments of the invention, the apatite cement is formed from an apatite-forming calcium-based precursor powder as described in any of the above embodiments, with the exception that the sodium pyrophosphate is provided in solution in an aqueous liquid with which the precursor powder is mixed to achieve hardening.

The composition may also include agents that facilitate a fast diffusion of water into the composition in situ, preferably non-ionic surfactants like Polysorbates. The amount of surfactant is preferably between 0.01 and 5 wt % of the powder composition, most preferably, 0.1-1 wt %.

In specific embodiments, salts may be dissolved into the liquid to obtain a faster or slower setting, e.g. citric acid, H₃C₆H₅O₇, sulfuric acid, H₂SO₄, and/or phosphoric acid, H₃PO₄. The hardening can then be performed in a dry environment.

In specific embodiments, the mean grain size of the precursor powder is preferably below 100 micrometer, and more preferably below 30 micrometer as measured in the volumetric grain size mode. Generally, smaller grain sizes give higher mechanical strength than larger grain sizes. In other embodiments, the grain size of the powders ranges from less than 100 micrometer up to about 600 micrometer, i.e., the precursor powder contains powders of varying sizes spanning the indicated range.

The apatite cement-forming compositions as described herein can be delivered prehardened in the form of granules, custom ceramic solid shaped implants, or ceramic tiles on metal or polymer meshes as disclosed in WO 2011/112145 A1, incorporated herein by reference, or on metal or polymer wires as disclosed in WO 2013/027175 A2, incorporated herein by reference. The apatite cement-forming compositions as described herein can be also be delivered as a premixed injectable material that sets and hardens in vivo.

In one embodiment, the apatite cement-forming compositions are delivered prehardened in the form of granules. In a specific embodiment, the granules have a size in a range of from about 100 μm to 5 mm, or, more specifically, from about 100 μm to 3 mm, or from about 100 μm to 1 mm. Such granules may be used in various implant applications, one example of which is for cleft repair.

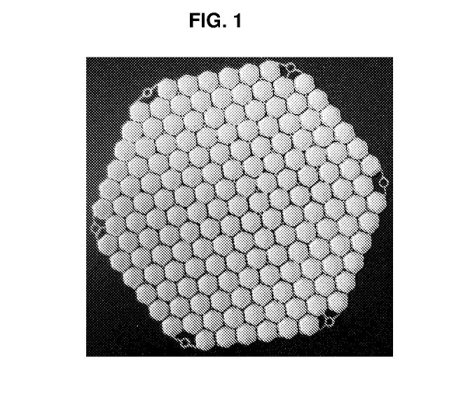

In one embodiment, in order to obtain a shapeable implant, the cement-forming compositions are moulded onto wires or mesh as shown in FIG. 1. Using a non-aqueous water-miscible liquid, using a mixture of water and a non-aqueous water-miscible liquid, or using only water, an apatite cement-forming composition as described herein is allowed to harden over portions of the wire or mesh to form an apatite cement mosaic implant, for example using a mould. In one embodiment, the cement-forming composition is hardened to form the apatite cement by placing the mould in a water-containing bath to expose the cement-forming composition to water. Once the cement is formed, the mosaic implant is released from the mould. After packing and sterilization, the mosaic implant is ready to be used. FIG. 1 shows a plurality of tiles formed of hardened hydraulic cement composition in a mosaic pattern moulded onto titanium mesh.

Implants formed of the apatite cement as described herein may be employed in methods for correcting or repairing bone defects. A specific embodiment comprises slowing implant resorption in a bone defect repair in a patient. The methods comprise providing the patient with an implant formed of an apatite composition as described comprising from 1 to 30 wt % of dicalcium pyrophosphate or sodium pyrophosphate, or, more specifically, β-dicalcium pyrophosphate. Advantageously, the resorption may be slowed such that less than 30%, less than 20% or less than 10% resporption occurs over a period of 6 months, 12 months, 18 months, 24 months, 30 months or 36 months, after implant in vivo.

Another specific embodiment comprises providing improved bone induction in a bone defect repair in a patient. These methods comprise providing the patient with an implant formed of an apatite composition as described comprising from 1 to 30 wt % of dicalcium pyrophosphate or sodium pyrophosphate, or, more specifically, β-dicalcium pyrophosphate. Advantageously, bone induction may be improved after implant in vivo.

Implants formed of the apatite cements as described herein may be employed in methods for slowing implant resorption and/or methods for improving bone induction in a bone defect repair in a patient, wherein the patient is provided with an implant formed of an apatite composition as described comprising from 1 to 30 wt % of dicalcium pyrophosphate or sodium pyrophosphate, or, more specifically, β-dicalcium pyrophosphate.

The specific embodiments set forth herein are illustrative in nature only and are not to be taken as limiting the scope of the invention defined by the following claims. Additional specific embodiments and advantages of the present invention will be apparent from the present disclosure and are within the scope of the claimed invention.

1. A calcium phosphate cement-forming composition, comprising an apatite-forming calcium-based precursor powder comprising α-tricalcium phosphate and/or tetracalcium phosphate and from 1 to 30 wt %, based on the weight of the precursor powder, of dicalcium pyrophosphate powder.

2. An apatite cement formed from the calcium phosphate cement composition according to claim 1 and, optionally, water.

3. An apatite cement comprising from 1 to 30 wt % of β-dicalcium pyrophosphate.

4. (canceled)

5. An implant comprising the apatite cement according to claim 3.

6. An implant comprising a titanium wire or mesh and one or a plurality of cement tiles moulded on the wire or mesh, wherein the cement tiles are formed of the apatite cement according to claim 3.

7. An implant according to claim 6, wherein the apatite cement comprises greater than 80 wt % apatite, 1 to 15 wt % β-tricalcium phosphate, and 1 to 15 wt % β-dicalcium pyrophosphate.

8-11. (canceled)

12. A method of correcting a bone defect in a patient by slowing in vivo implant resorption in a bone defect repair in a patient, the method comprising implanting an implant according to claim 5 in the patient.

13. A method of correcting a bone defect in a patient by providing in vivo improved bone induction in a bone defect repair in a patient, the method comprising implanting an implant according to claim 5 in the patient.

14-15. (canceled)

16. An apatite cement according to claim 3, comprising from 1 to 15 wt % of β-dicalcium pyrophosphate.

17. An apatite cement according to claim 3, comprising from 5 to 15 wt % of β-dicalcium pyrophosphate.

18. An apatite cement according to claim 3, comprising from 5 to 20 wt % of β-dicalcium pyrophosphate.

19. An apatite cement according to claim 3, comprising from 1 to 10 wt % of β-dicalcium pyrophosphate.

20. The implant according to claim 5, in the form of granules.

21. An implant comprising the apatite cement according to claim 16.

22. An implant comprising the apatite cement according to claim 17.

23. An implant comprising the apatite cement according to claim 18.

24. An implant comprising the apatite cement according to claim 19.

25. A method according to claim 12, wherein less than 30% resorption of the cement occurs 6 months after implantation.

26. A method according to claim 12, wherein less than 20% resorption of the cement occurs 6 months after implantation.
